(19) United States
(12) Patent Application Publication
(10) Pub. No.: US 2022/0395190 A1
(43) Pub. Date: Dec. 15, 2022

(54) Deployment of Sensors

(71) Applicant: Foundry Innovation & Research 1, Limited, Dublin (IE)

(72) Inventors: Fiachra M. Sweeney, Dublin (IE); Stephen Sheridan, Dublin (IE); Deirdre McGowan, Dublin (IE)

(21) Appl. No.: 17/771,317

(22) PCT Filed: Oct. 23, 2020
(86) PCT No.: PCT/EP2020/079939

§ 371 Date: Apr. 22, 2022

Related U.S. Application Data
(60) Provisional application No. 62/925,842, filed on Oct. 25, 2019.

(51) Int. Cl. A61B 5/07; A61B 5/107; A61B 5/02

(52) U.S. Cl. CPC A61B 5/076; A61B 5/1076; A61B 5/02007

(57) ABSTRACT

A system is provided for deployment of a sensor in a blood vessel comprising: an expandable sensor; at least one anchor element attached to the sensor; an expandable element configured within the sensor such that, in use, expansion of the expandable element causes the sensor to radially expand to fix the at least one anchor element in a wall of the blood vessel.

### FIELD

The present disclosure is directed to the deployment of sensors in blood vessels. In particular, it is directed to the deployment and anchoring of wireless vascular monitoring implants.

### BACKGROUND

Heart failure, also often referred to, as congestive heart failure, occurs when the myocardium cannot efficiently provide oxygenated blood to the vascular system. A variety of pathophysiological conditions, such as myocardial damage, diabetes mellitus, and hypertension gradually disrupt organ function and auto regulation mechanisms, leaving the heart unable to properly fill with blood and eject it into the vasculature. In parallel, heart failure can interact unfavourably with a series of complications such as heart valve problems, arrhythmias, liver damage and renal damage or failure.

There have been previous attempts to develop vascular monitoring devices and techniques, including those directed at monitoring vessel arterial or venous pressure or vessel lumen dimensions.

However, many such existing systems are catheter based (not wireless) and thus can only be utilized in a clinical setting for limited periods of time, and may carry risks associated with extended catheterization.

As such, new developments in the field of sensor deployment are in order to provide doctors and patients with reliable and affordable wireless vascular monitoring implementation, particularly in the critical area of heart failure monitoring.

### SUMMARY

The present disclosure provides a system for deployment of a sensor in a blood vessel comprising:
an radially expandable sensor;
at least one anchor element attached to the sensor, the anchor element being configured to engage the vessel wall and maintain the sensor in contact therewith during physiologic expansion and contraction of the vessel;
an expandable element configured within the sensor such that, in use, expansion of the expandable element causes the sensor to expand to radially fix the at least one anchor element in a wall of the blood vessel,
wherein the sensor is configured to produce a wireless signal correlated with vessel diameter or area.

This is advantageous as it provides for deployment of a sensor within a vessel wherein the sensor is not sufficiently radially resilient to rely on self-expansion for anchoring. The expandable element serves to expand the non-resilient sensor to the extent that it can contact and fix itself to a vessel wall via an anchor element. Once fixed in place, the anchor element serves to maintain the sensor in contact therewith during physiologic expansion and contraction of the vessel. As such, the sensor may produce a wireless signal correlated with vessel diameter or area as the diameter or area change during expansion and contraction of the vessel.

The anchor may comprises an erodible or fracturable element configured such that, upon fixing of the at least one anchor element in the wall of the blood vessel, erosion or fracture of the element decouples the sensor from the anchor. Furthermore, the erodible element may be biodegradably erodible.

This is advantageous as it provides the ability for the sensor to be mechanically decoupled from the anchor at a time post deployment, potentially when the sensor and anchors have healed into the vessel. As such, the decoupling could be achieved via a biodegradable portion that is designed to erode over time in vivo or via a section that is designed to intentionally fracture either due to physiological loading or via an applied stimulus. This stimulus could be either an internal mechanical force such as re-expansion using a balloon or other mechanical device or via the application of focussed energy such as ultrasound from either within the vessel or from outside the body.

Additionally, the anchor may be fracturable/degradable at locations around the circumference of the sensor. This provides that the anchor may start as a complete ring then fracture over time after it has grown into the vessel. This can allow for sensors or markers to be placed on opposing sides of a vessel. Furthermore, a ring or coil type sensor may be rigid upon initial deployment. A coating or sheath on the sensor may degrade over time to allow it to become more flexible.

The at least one anchor element may be moveable from a first position wherein the anchor is radially inward and non-engageable with the vessel wall (i.e. attached to the sensor which is crimped within or onto a delivery system or the anchor element is retained in a crimped position by an anchor retaining sheath) to a second position wherein the anchor is located radially outward so as to be engageable with the vessel wall (i.e attached to the sensor which is expanded via a balloon or other mechanical system, or the retraction of an anchor retaining sheath). This is advantageous as it provides that the sensor can be manoeuvred through the vasculature into position for anchoring without the risk of the anchor inadvertently fixing in the wall of the blood vessel in an undesired location. The anchor can be moved to the second position only when it is desired to fix the anchor and thus the sensor in place. Certain embodiments may also provide more control of this deployment procedure.

The system may further comprise a sheath configured at least partially about the sensor, the sheath and anchor element configured for protecting the expandable element from the at least one anchor element. In this manner, the sheath may fully surround the sensor and the expandable element and aids in the delivery of the expandable element and sensor into a vessel. Alternatively, the sheath may be configured to surround the expandable element, wherein the expandable element and the sheath are positioned within the sensor, for example along the horizontal axis of a coil shaped sensor. In this manner, the sheath is positioned between the expandable element and the sensor and thus protects the surface of the expandable element from being damaged by the anchors of the sensor. This is advantageous as it provides that the expandable element, which may be made from a fragile or tearable material is shielded from the anchor, which may be of a jagged or sharp configuration.

The sheath may be removable from about the sensor such that removal of the sheath moves the anchor element from the first non-engagable position to the second enagagable position. This is advantageous as it provides for a reliable method of enclosing the anchor until it is a position where it is desired to fix the anchor and thus the sensor in place. Removal of the sheath at this time provides the dual advantage of protecting the expandable element until the anchor is to be fixed and further providing for an effective means of moving the anchor from the first position wherein the anchor is non-engageable with the vessel wall to the second position wherein the anchor is engageable with the vessel wall. This provides that non resilient sensors or sensors that are not sufficiently radially resilient may be expanded radially by the expandable element to a position such that the sensor is in contact with or in close proximity to the inner wall of a vessel. Removal of the sheath at this time provides that the anchor is released or deployed into an engageable configuration such that it may fix to the vessel wall.

The movement of the anchor is in effect caused by the positioning of the sheath. The anchor element may be manufactured and configured in such a manner as to be provided on the sensor in an orientation to facilitate engagement with the vessel wall and retained in this orientation by the positioning of the sheath. As such, any unintended engagement of the anchor with the vessel wall is prevented by positioning the sheath around or about the anchor.

The expandable element may be a balloon. The balloon approach is advantageous as a balloon provides for a reliable method of expanding a non-resilient expandable sensor. Variations in balloon shape can provide for deployment of sensor into a range of different vessel internal shapes and sizes and also provides the ability to apply different forces to different sections of the implant. Use of a balloon to expand the sensor provides that the overall profile of the sensor prior to deployment can be reduced providing that deployment of the sensor into a vessel is as minimally invasive as possible. The expandable element may also comprise a mechanical device or other mechanical expansion system.

The expandable element of the system may comprise a tube. The tube may comprise non-rigid material. This is advantageous as is provides for ease of insertion into a vessel and further provides that a force applied to the tube material may serve to change the shape of the tube.

The tube may comprise a plurality of incisions through the surface of the tube along its length. This provides a region of the tube which can respond by changing shape upon the application of a force to the tube.

The incisions may be straight, curved, “S” shaped or the incisions may be saw-tooth shaped. This has the effect of controlling the extent of the change of shape of the tube upon the application of a force to the tube.

A region of the tube around the incisions may be expandable upon application of a force to the tube. Providing that the tube may be expandable by application of a force in this manner provides that, when the tube is placed within a vessel, a portion of the outer surface of the tube may be moved closer to the inner surface of the vessel. As such, a sensor placed around the outer surface of the tube may be moved closer to and ultimately make contact with the inner surface of a vessel.

Application of a force to the tube may comprise applying a longitudinal pulling force to an internal surface of the tube. The system may further comprise a tether element fixed to an internal surface of the tube. A longitudinal pulling force may be applied to the tether. This provides for a simple method of applying a sufficient force to the tube to cause to region of the tube comprising the incisions to expand.

The application of a force to the tube may comprise applying a longitudinal pushing force to an external surface of the tube. The tube may comprise a first inner tube and a second outer tube. The longitudinal pushing force may be applied to the second outer tube. This provides for an additional simple method of applying a sufficient force to the tube to cause to region of the tube comprising the incisions to expand.

The at least one anchor element may comprise a point, a harpoon, a fish hook, a barb, scales or a corkscrew shape. Each of the anchor elements can serve to securely fix the sensor in place within a vessel. Typically, the anchor element will embed itself into the vessel wall to hold it in position. This embedding may be at a macro feature or micro or surface feature level. Each described element provides a unique anchoring mechanism. A simple point at an angle provides resistance to extraction in a specific direction, a harpoon provides the advantage of resistance against extraction. A fish hook provides the advantage of ease of insertion. A corkscrew provides the advantage of depth of insertion.

The sensor may be a ring or coil shaped and the at least one anchor element may be attached to a first end of the sensor. This is advantageous as the anchor element is provided in a position such that expanding the expandable element has the effect of moving the anchor element towards the vessels wall for fixing in the wall.

Other sensor embodiments may involve active or passive reflectors, and utilise energy sources such as ultrasound, impedance or inductance, which may be retained in position in the lumen using at least one anchor element attached to the sensor.

The system may further comprise at least a second anchor element wherein the at least second anchor element is attached to a second end of the sensor, opposite the first. This is advantageous as the first and second anchor elements are provided in positions such that expanding the expandable element has the effect of moving the anchor element towards the vessel walls in opposing directions such that first anchor may be fixed in one side of a vessel while the second anchor may be fixed in an opposite side of the vessel or at a longitudinally distant location within the vessel.

The system may further comprise an anchor element attached to the sensor between the first and second end of the sensor. This is advantageous as it provides for additional anchoring of the sensor in addition to being anchored at each of its ends.

Alternative embodiments may have multiple anchor elements located circumferentially around a cylindrical or coil shaped sensor. This is advantageous as it provides for additional fixation along the length of the sensor.

The sensor may be configured to obtain a diameter or area measurement of the blood vessel. This is advantageous as diameter or area measurements can be utilised to ascertain physiological information about the vessel and ultimately the patient.

The blood vessel may be a vein or artery. The vein may be one of the jugular vein, the superior vena cava or the inferior vena cava, IVC. This is advantageous as measurements from such vessels provide indicators as to vascular health.

The present disclosure further provides a method for deployment of a sensor in a blood vessel comprising:
inserting a radially expandable sensor into a blood vessel, the sensor comprising at least one anchor element;
expanding an expandable element configured within the sensor such that expansion of the expandable element causes the sensor to expand to radially fix the at least one anchor element in a wall of the blood vessel,
removing the expandable element from within the sensor; and
receiving a wireless signal correlated with diameter or area of the blood vessel from the sensor;
wherein the anchor element maintains the sensor in contact with the vessel wall during physiologic expansion and contraction of the blood vessel.

The method may further comprise removing a sheath from the sensor prior to expanding the expandable element. The blood vessel may be a vein. The vein may be one of the jugular vein, the superior vena cava or the inferior vena cava, IVC.

### BRIEF DESCRIPTION OF THE DRAWINGS

FIG. 1 is a schematic plot of patient fluid volume versus response employing IVC diameter or area measurement (curves A1 and A2) in comparison to prior pressure-based systems (curve B) and in general relationship to IVC collapsibility index (IVC CI, curve C).

FIG. 2 shows a measurement being obtained from a patient via a remote monitoring system

FIG. 3 shows a schematic example of a sensor which can be used according to the system of the present disclosure

FIG. 4 shows an example of a sensor which can be used according to the system of the present disclosure

FIG. 5 shows a close up of a sensor surface

FIG. 6 shows a deployment system according to the present disclosure prior to expansion of the expandable element

FIG. 7 shows a deployment system according to the present disclosure after expansion of the expandable element

FIG. 8 shows an expandable element in the form of tubing with a series of incisions

FIG. 9 shows a further example of an expandable element in the form or tubing with a series of incisions

FIG. 10 shows the expandable element of FIG. 8 after expansion of the expandable element

FIG. 11A shows the expandable element of FIG. 8 configured to deploy a sensor prior to expansion of the expandable element. FIG. 11B shows the expandable element of FIG. 8 configured to deploy a sensor after expansion of the expandable element

FIG. 12 shows an expandable element in the form of tubing with a tether for expansion of a cut region of the tubing

FIG. 13 shows an expandable element in the form of an inner tube and an outer tube

FIG. 14A shows the sheath in a positon surrounding the expandable element and sensor.

FIG. 14B is a close up view of the sheath, expandable element and sensor.

FIG. 15A shows the sheath retracted away from the expandable element and sensor. FIG. 15B is a close up view of the sheath, expandable element and sensor.

FIG. 16A shows an anchor in a non engageable state while FIG. 16B shows an anchor in an engageable state

FIG. 17 shows a sensor fixed in place with anchors after deployment in a vessel

FIG. 18 shows a sensor with a number of anchor elements

FIG. 19 shows a sensor and anchor element joined by a plurality of erodible or fracturable elements.

FIG. 20A-FIG. 20H show a number of example anchor elements types.

### DETAILED DESCRIPTION

Use of Area Measurements of Blood Vessels

The assignee of the present disclosure has developed a number of devices that provide fluid volume data based on direct measurement of physical dimensions of blood vessels such as the diameter or area. Examples of these devices are described, for example, in PCT/US2016/017902, filed Feb. 12, 2016, and WO2018/031714, filed Aug. 10, 2017 by the present Applicant, each of which is incorporated by reference herein in its entirety. Devices of the types described in these prior disclosures facilitate new management and treatment techniques based on regular intermittent (e.g., daily) or substantially continuous (near real-time), direct feedback on physical dimensions of blood vessels.

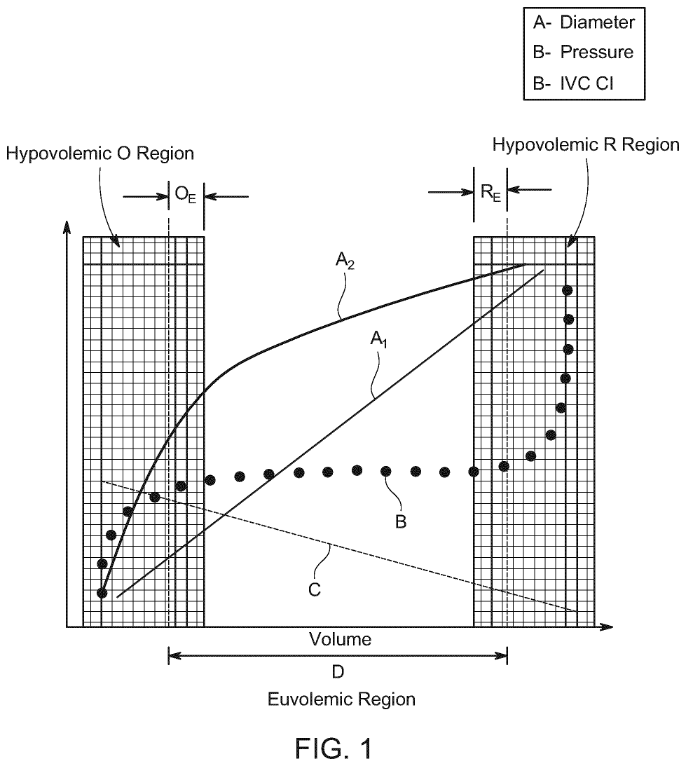

WO2018/031714 further describes some of the advantages of the information that can be derived from taking area type measurements using these devices. As can be seen in FIG. 1 (reproduced from FIG. 1 of WO2018/031714), the response of pressure-based diagnostic tools (B) over the euvolemic region (D) is relatively flat and thus provides minimal information as to exactly where patient fluid volume resides within that region. Pressure-based diagnostic tools thus tend to only indicate measureable response after the patient's fluid state has entered into the hypovolemic region (O) or the hypervolemic region (R). In contrast, a diagnostic approach based on diameter or area measurement across the respiratory and/or cardiac cycles (Ai and A₂), which correlates directly to r C volume and IVC CI (hereinafter “IVC Volume Metrics”) provides relatively consistent sensitive information on patient fluid state across the full range of states.

It is noted in WO2018/031714 that using vessel area measurement, in this example with respect to the inferior vena cava (IVC), as an indicator of patient fluid volume provides an opportunity for earlier response both as a sensitive hypovolemic warning and as an earlier hypervolemic warning. With respect to hypovolemia, when using pressure as a monitoring tool, a high pressure threshold can act as a potential sign of congestion, however when pressure is below a pressure threshold (i.e., along the flat part of curve B), it gives no information about the fluid status as the patient approaches hypovolemia. With respect to hypervolemia, vessel area measurements, for example potentially provide an earlier signal than pressure-based signals due to the fact that IVC diameter or area measurements change a relatively large amount without significant change in pressure. Hence, a threshold set on IVC diameter or area measurements can give an earlier indication of hypervolemia, in advance of a pressure-based signal.

Obtaining Area Measurements of Blood Vessels

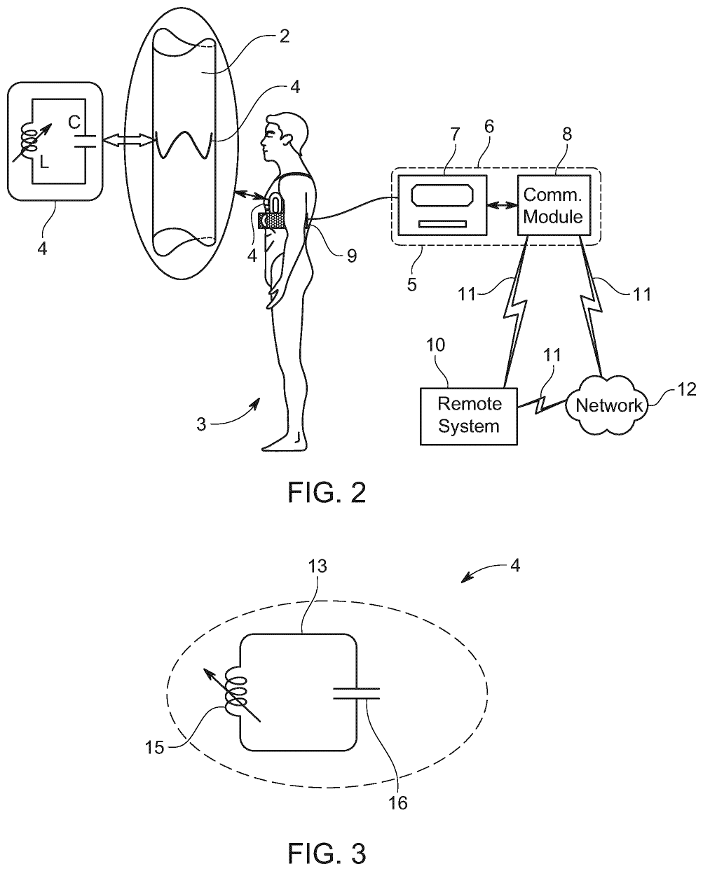

Systems and sensors for obtaining area measurements of blood vessels are described in WO2018/031714. While the examples therein are described with respect to obtaining measurements from the IVC, the sensors described may be utilised for obtaining measurements from other vessel types, for example from the jugular vein, the superior vena cava and other vessel types. FIG. 2 shows aspects of such a system 1 for obtaining measurements from the IVC 2 of a patient 3 utilizing a sensor 4.

A processor 5 may take the form of a laptop or desktop computer. The processor 5 may further be a mobile telecommunication device such as a mobile telephone or tablet. The processor may further be a wearable electronic device or sensor reader. In the case that the processor is incorporated into the sensor reader, the reader shall be capable of wirelessly transmitting and receiving the required radiofrequency pulses, filtering and processing them as required and operating the appropriate software for interpreting the results. The processor is configured with suitable software for interpretation of the sensor measurements. The sensor 4 and processor 5 may in some embodiments be further configured to communicate with control and communications modules, and one or more remote systems such as processing systems, user interface/displays, data storage, etc., communicating with the control and communications modules through one or more data links, preferably remote/wireless data links. FIG. 2 shows aspects of such systems. Such a system may include a control module 6 to communicate with and, in some embodiments, power or actuate the sensor. The sensor may thus be remotely powered by the control module. In an alternative embodiment, the sensor may be internally powered without the requirement for a remote power source. The processor may be comprised within the control module 6. Alternatively, the processor 5 may be as a separate device. Control module 6 may include controller 7 and communications module 8. The control module may comprise a bedside console. For patient comfort, as well as repeatability in positioning, a belt reader or antenna 9 may be worn by the patient around the waist. The antenna may serve to wirelessly transmit measurements from the sensor 4 to the processor 5. Information may be transferred 11 from the communications module 8 via Bluetooth, wi-fi, cellular, or local area network to a remote system 10 and/or to a network 12 for storage and/or further analysis.

The sensor 4 may take the form of an implantable device. To obtain measurements, sensor is implanted into a blood vessel using the deployment system as described further below. Once in position and activated, a sensor is capable of obtaining modulating area measurements from the vessel via modulations in their inductance and therefore frequency. The processor obtains the measurements from a sensor by, for example, wireless link to or resonant coupling with the sensor. Once obtained by the processor, the measurements are processed and analysed as set out in further detail below to determine the dimensions of the blood vessel.

Measurements of vessel diameter or area by the sensor 4 may be made continuously over one or more respiratory cycles to determine the variations in vessel dimensions over this cycle. Further, these measurement periods may be taken continuously, at preselected periods and/or in response to a remotely provided prompt from a signal within the system or from a health care provider/patient.

The first sensor 4 may employ a variable inductance L-C circuit 13 for performing measuring or monitoring functions described herein, as shown schematically in FIG. 3. The sensor 4 may also include anchor element for securely anchoring within the IVC or other vessel. The deployment and anchoring of the sensor will be described in more detail below. Using a variable inductor 15 and known capacitance 16, L-C circuit 13 produces a resonant frequency that varies as the inductance is varied. Changes in shape or dimension of the vessel cause a change in configuration of the variable inductors, which in turn cause changes in the resonant frequency of the circuits.

Thus, not only should the sensor be securely positioned at a monitoring position, but also, at least a variable coil/inductor portion 13 of the implant may have a predetermined compliance (resilience) selected and specifically configured to permit the inductor to move with changes in the vessel wall shape or dimension while maintaining its position with minimal distortion of the natural movement of the vessel wall. Thus the variable inductor may be specifically configured to change shape and inductance in proportion to a change in the vessel shape or dimension.

Because the sensor does not rely on resilience or outward bias to provide anchoring, instead being held in place by anchor elements 14, coil/inductor portion 13 may be extremely flexible and compliant so that it has minimal impact on the natural expansion and contraction of the vessel.

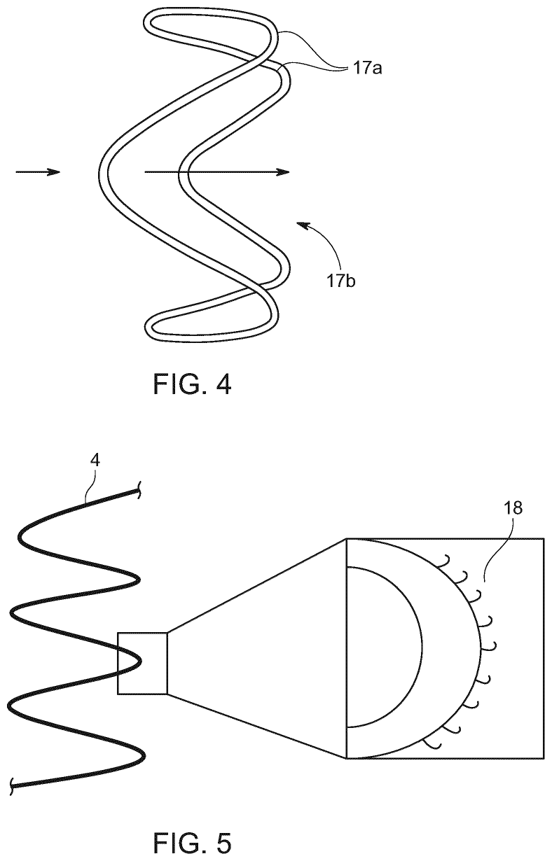

Variable inductor 15 is configured to be remotely energized by an electric field delivered by one or more transmit coils within antenna module 9 positioned external to the patient. When energized, L-C circuit 13 produces a resonant frequency which is then detected by one or more receive coils of the antenna module. Because the resonant frequency is dependent upon the inductance of the variable inductor, changes in shape or dimension of the inductor caused by changes in shape or dimension of the vessel wall cause changes in the resonant frequency. The detected resonant frequency is then analysed by the processor component of the system to determine the vessel diameter or area, or changes therein. FIG. 4 shows an example of a sensor type which may be deployed in accordance with the present disclosure. The sensor comprises a ring shaped coil having a sinusoidal or zig-zag shape including a number of crowns 17a linked by a number of struts 17b.

Sensor materials may include metals such as stainless steel, cobalt chromium, platinum or tantalum alloys; and polymers such as PLLA (Poly L Lactic Acid). Features of sensor embodiments disclosed herein are that they exert very low radial force, are expanded via the delivery system and are retained in contact with the vessel wall via anchors as it moves through cycles of distension and collapse.

The sensor may further be composed of wire required for electrical transmission within the sensor and the anchor elements. These anchor elements may be surface characteristics of a polymer coating on the sensor wire. The surface may comprise micro or nano-scale hooks that catch the vessel wall on contact. FIG. 5 shows a close up of a sensor 4 surface wherein the anchor element comprises a plurality of hooks 18. The hooks or barbs may be micrometres or nanometres in dimension. Other shapes could also be used to ensure high compliance (low radial force) such as coil or spiral embodiments.

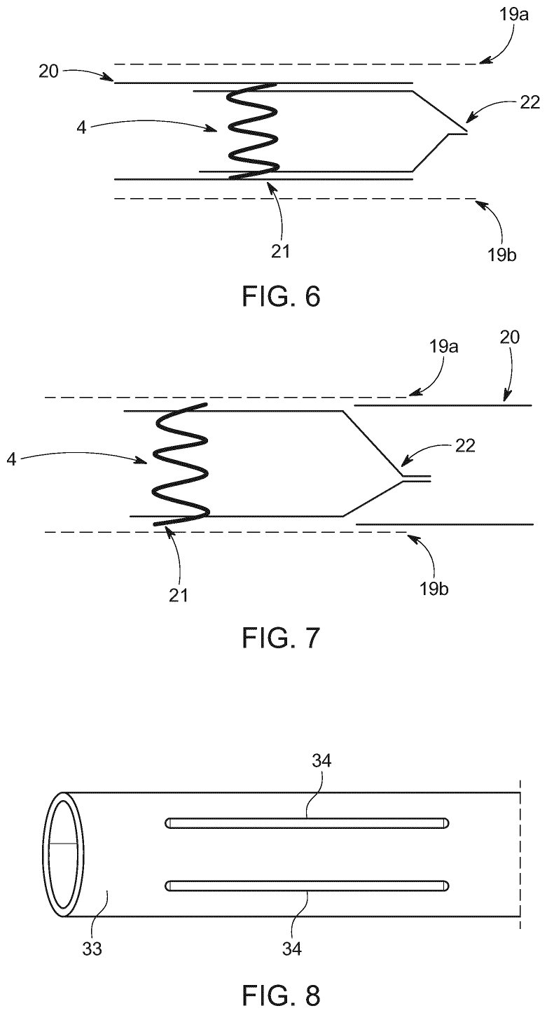

FIG. 6 shows a system for deployment of a sensor in a blood vessel according to the present disclosure. The system is shown within the walls 19 a,b of a vessel (indicated as dashed lines). The vessel may be an artery or vein. Veins such as the jugular vein, the superior vena cava, the inferior vena cava, IVC are specific targets. The system comprises a non-resilient expandable sensor 4. The sensor may be of the type shown in FIG. 4 or an alternative type. A coil type sensor may be used. A removable sheath 20 about the sensor is also shown and this may be withdrawn proximally or distally. Plural anchor elements 21 are attached to the sensor. An expandable element 22 is configured within the sensor such that expansion of the expandable element 22 causes the sensor 4 to expand to fix the at least one anchor element 21 in a wall of the blood vessel. FIG. 6 shows the expandable element is a non-expanded state. Likewise, the sensor is in a non-expanded state. The expandable element is shown as a balloon type expandable element. The balloon is configured to pass “through” the sensor, for example as per the horizontal arrows through the centre of the sensor as shown in FIG. 4. In this manner, expansion of the balloon will cause the non-resilient expandable sensor to expand as the balloon will apply pressure to the inner surfaces of the sensor and tend to “push” it apart causing the sensor to expand within the vessel. Expansion of the balloon can be stopped once the ends of the sensor are close to or in contact to the inner walls of the vessel. (FIG. 7 shows the balloon expanded such that the sensor is in much closer proximity to the inner walls of the vessel than when the balloon is in a non-expanded state). The sheath may be removed as described below once a satisfactory position for the sensor is found.

The anchor elements may be moveable from a first position wherein the anchor element is non-engageable with the vessel wall to a second position wherein the anchor element is engageable with the vessel wall. In FIG. 6, the anchor element is configured in the non-engageable position during the period when the sensor is crimped on the balloon and protected by the sheath and in the engageable position (FIG. 7) while the sensor is being expanded by the expandable element and the sheath retracted.

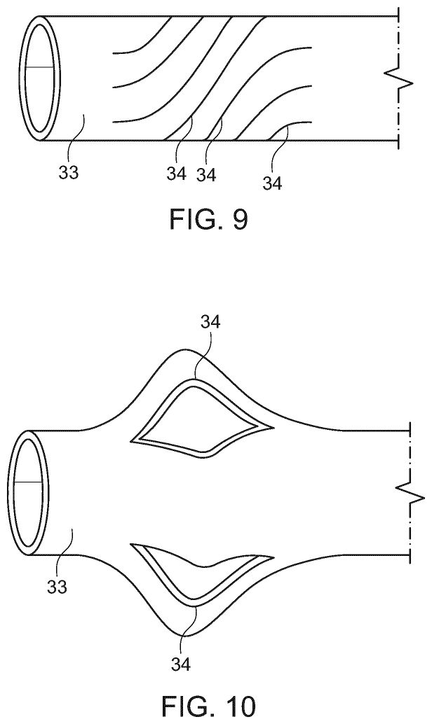

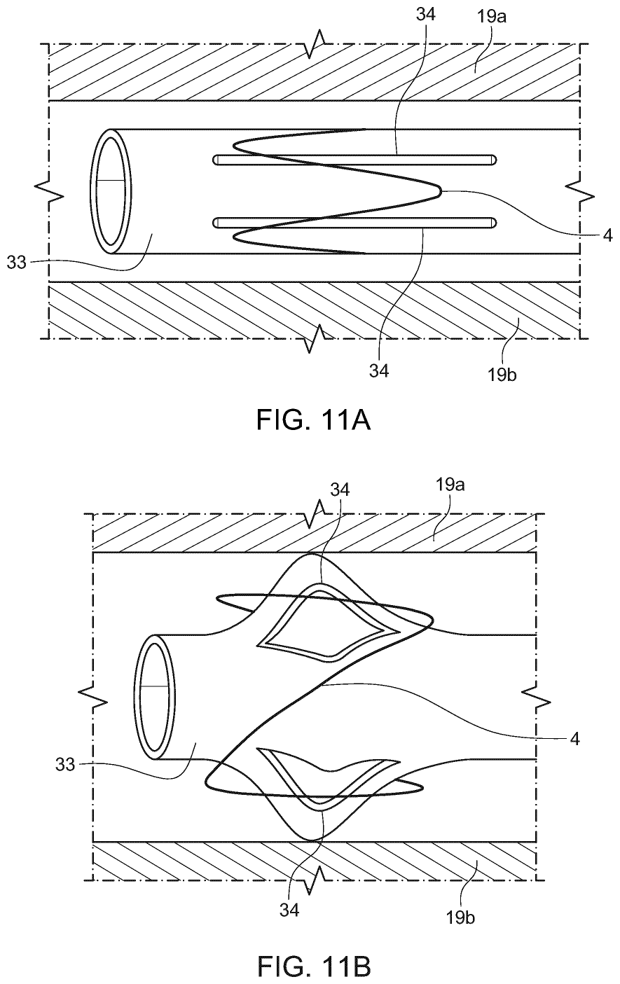

FIGS. 8 to 13 show an alternative to the balloon type expandable element 21. FIG. 8 shows an expandable element in the form of a piece of tubing 33, for example catheter tubing, with a series of longitudinal cuts or incisions 34 through its surface. The incisions 34 are provided radially around the circumference of the tubing. Alternative patterns of incisions may be provided. For example, FIG. 9 shows a series of “S” or wave shaped incisions. Providing incisions through the surface in this manner has the effect of causing the tubing 33 to expand (FIG. 10) around the incision region 35 when the tubing is compressed longitudinally. As such, the tubing 33 as described may be configured within a sensor 4 (FIG. 11A) such that expansion of the incision region 35 causes the sensor 4 to expand (FIG. 11B) to fix the at least one anchor element in a wall 19a, 19b of the blood vessel.

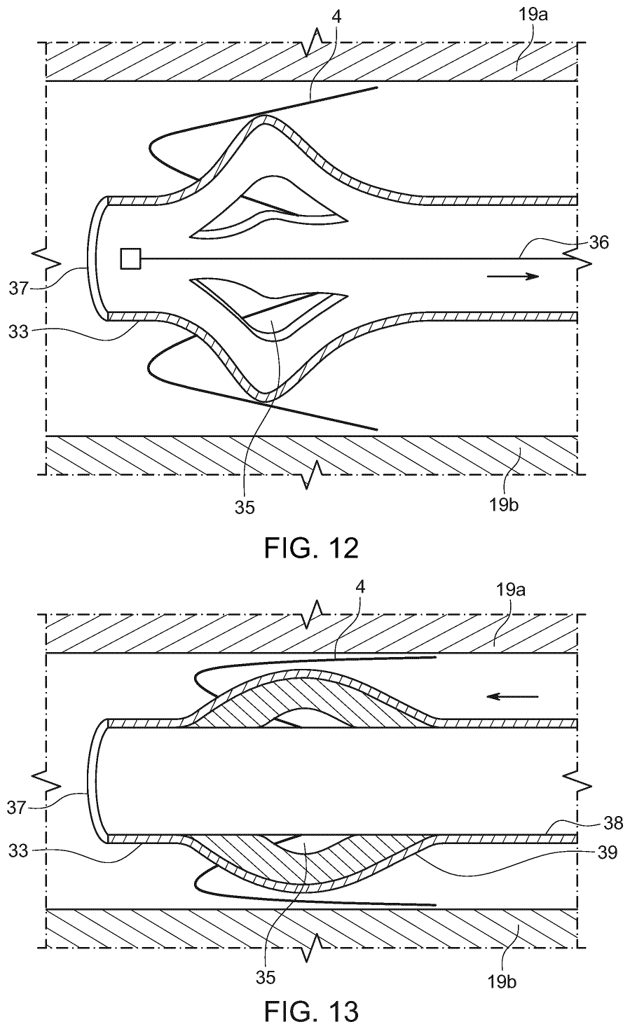

The tubing 33 may be compressed longitudinally by applying a longitudinal force through the tubing. A number of alternative arrangements may be provided in order to apply the required force for expanding the tubing. A tether 36 may be provided and attached to an internal surface at the head or tip 37 of the tubing (FIG. 12). Thus, if the tubing is held in place and a pulling force is applied to the tether, the tether 36 is retracted through the tubing and the tip 37 is caused to be pulled back towards the incision region 35. This causes the incision region 35 to compress longitudinally and has the effect of causing the incision region to expand outwards.

Alternatively, the tubing 33 may be provided as a first inner tube 38 surrounded by a second outer tube 39 (FIG. 13). The inner tube 38 is bonded to the outer tube 39 towards the head or tip 37 of the tubing. The incision region 35 is provided on the outer tube. The inner tube may act as a guide wire lumen. With the inner tube remaining stationary, a pushing force is applied to the outer tube and thus the outer tube 39 is pushed forward in the direction of the tip 37. As the inner and outer tubes are bonded towards the tip, the pushing action causes the incision region to compress longitudinally and has the effect of causing the incision region to expand outwards. Again, in such an arrangement, if the tubing 33 is configured within a sensor 4 such that expansion of the incision region 35 causes the sensor 4 to expand, this will have the effect of fixing at least one anchor element in a wall 19a, 19b of the blood vessel.

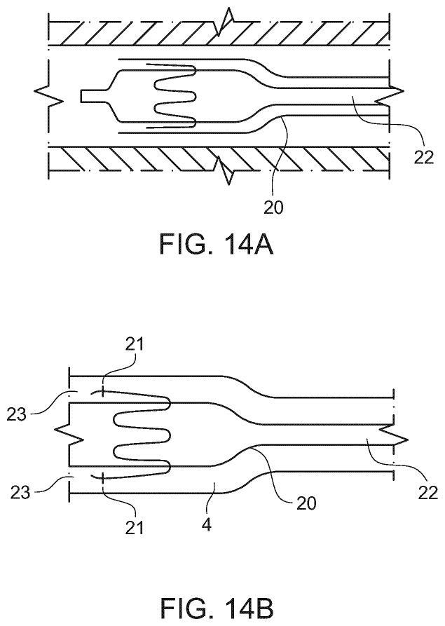

FIG. 14A shows the sheath 20 in a positon surrounding the expandable element 22 and sensor. FIG. 14B is a close up view of the sheath 20, expandable element 22 and sensor 4. This shows the anchor element 21 configured in the non-engageable position with prongs 23 on the anchor element 21 facing away from the wall of the vessel.

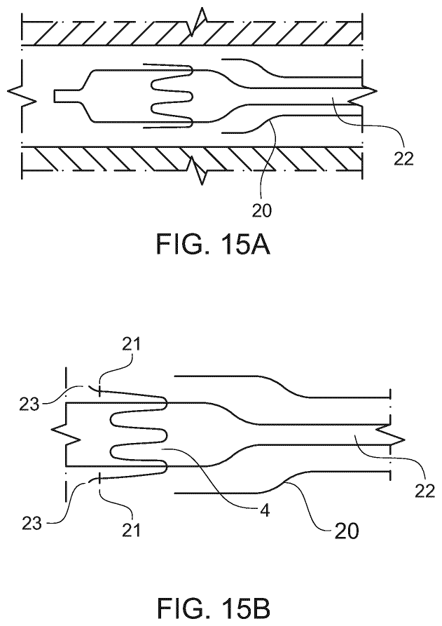

FIG. 15A shows the sheath 20 retracted away from the expandable element 22 and sensor. FIG. 15B is a close up view of the sheath 20, expandable element 22 and sensor 4. This shows the anchor element 21 configured to be engageable with the vessel wall with prongs 23 on the anchor element 21 facing towards the wall of the vessel.

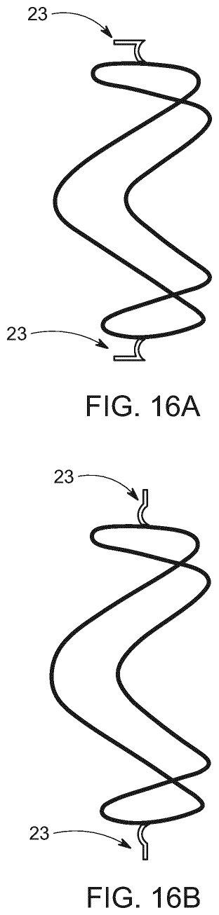

FIG. 16A shows an anchor element in a non-engageable state wherein prongs 23 of the anchor element are flattened, potentially through interaction with the sheath, so that they would present a horizontal surface to an inner wall of the sheath and thus not be primed for engagement or fixing in the wall. FIG. 16B shows an anchor element in an engageable state wherein prongs of the anchor 23 are upright, following the removal of the sheath, so that they would present a vertical surface to an inner wall of the vessel and thus would be primed for engagement or fixing in the wall. In one example, prongs 23 are deflectable by the sheath into the configuration of FIG. 16A and is resiliently biased into the configuration of FIG. 16B when the sheath is retracted. Furthermore, the prongs 23 may be transformed from a retracted position to a radially protruding position by expansion of the balloon and/or the change in size or geometry of the sensor.

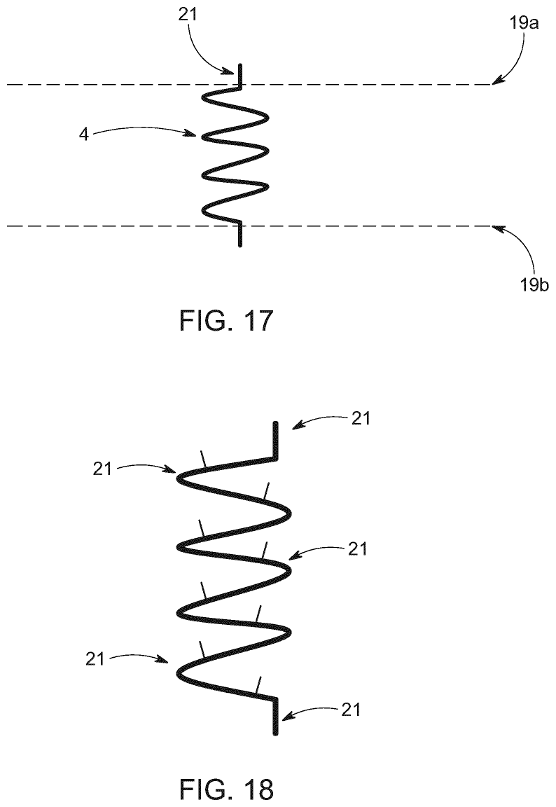

Once a satisfactory position in a vessel for the sensor has been selected, the sheath may be removed from about the sensor such that removal of the sheath moves the anchor element from the first position to the second position. The balloon is then expanded to deploy the sensor and force the anchors into the vessel wall. The balloon can then be deflated and removed. Upon removal of the balloon and the sheath, the sensor remains anchored in place with the anchors fixed into the vessel walls 19 a, b (FIG. 17).

FIGS. 16A and 16B show an anchor element peripherally attached on a ring shaped sensor on opposing sides of the sensor. An anchor element is attached to a first side of the ring and a second anchor element is attached to an opposing side of the ring. In such an arrangement, with two opposing anchors, it is advantageous for the anchors to be located on the anterior 12 o'clock and posterior 6 o'clock positions on the sensor so that they may move with the anterior and posterior walls of the vessel. However, other arrangements of sensor shape and anchor positions are possible. For example, a coil type sensor may be extended across the diameter of a vessel such that a first end of the sensor is in contact with a first side of the wall of the vessel and a second opposite end of the sensor is in contact with an opposing side of the wall of the vessel. In this arrangement, an anchor may be provided at each end of the sensor wherein an anchor element is attached to the first side of the sensor and a second anchor element is attached to the second opposing side of the sensor, opposite the first. FIG. 18 shows a sensor with a number of additional anchor elements 21 attached at spaced apart locations around the circumference of the sensor. With reference back to the sensor of FIG. 4, anchors may be placed, for example, in multiple positions on either the crowns 17a or struts 17b or a combination thereof.

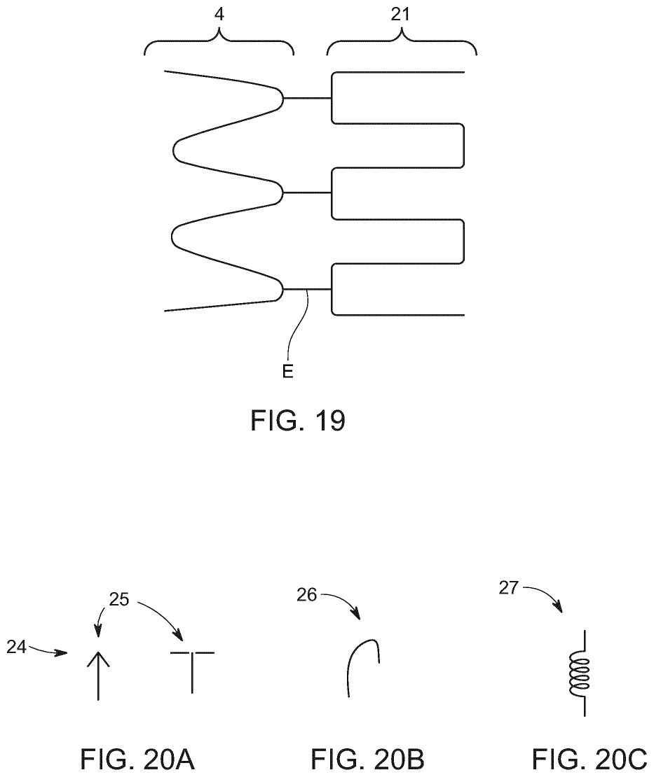

FIG. 19 shows a sensor 4 and anchor element 21, the sensor being joined to the anchor by a plurality of erodible or fracturable elements (E). Upon fixing of the anchor element in the wall of the blood vessel, erosion or fracture of the element decouples the sensor from the anchor, for example, at such a time when the sensor and anchors have healed into the vessel

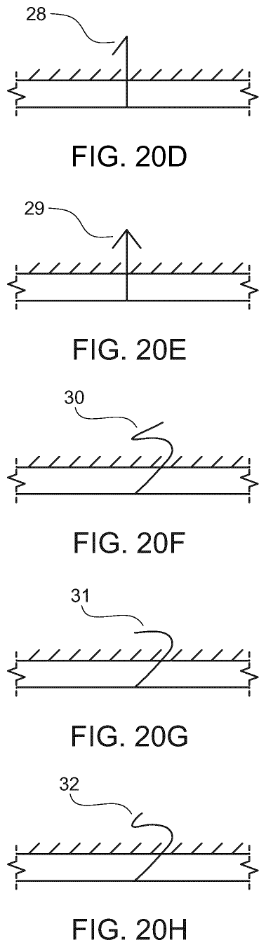

FIG. 20A—FIG. 20H show a number of example anchor elements which may be used in accordance with the present disclosure. FIG. 20A shows a harpoon 24 type anchor with end points 25 moveable between a first position (left) upon insertion into a vessel wall and a second position (right) to retain the anchor in the vessel wall. FIG. 20B shows a fish hook 26 type anchor while FIG. 20C shows a corkscrew 27 type anchor. FIG. 20D shows a “half arrow” type anchor 28 while FIG. 20E shows a “full arrow” 29. FIG. 20F shows a curved anchor element with a half arrow tip 30. FIG. 20G shows a curved anchor element with a pointed tip 31. FIG. 20H shows a curved anchor element with a pointed tip 32 curved away from the anchor base.

Individual anchors may have anchor length/depths of up to 3-5 mm to avoid perforation and yet provide anchoring.

Further provided is a method for deployment of a sensor in a blood vessel. The sensor may be any of the sensor types with anchor configurations as described above. The method comprises inserting a radially expandable sensor into a blood vessel, the sensor comprising at least one anchor element. Once the sensor is in position in a vessel, the method comprises expanding an expandable element configured within the sensor such that expansion of the expandable element causes the sensor to expand to fix the at least one anchor element in a wall of the blood vessel.

Upon being fixed in position in a vessel as described with respect to the system and method of the disclosure, the sensor is ready to perform measurements such as the area measurements described herein.

The words “comprises/comprising” and the words “having/including” when used herein with reference to the present disclosure are used to specify the presence of stated features, integers, steps or components but do not preclude the presence or addition of one or more other features, integers, steps, components or groups thereof.

It is appreciated that certain features of the disclosure, which are, for clarity, described in the context of separate embodiments, may also be provided in combination in a single embodiment. Conversely, various features of the disclosure which are, for brevity, described in the context of a single embodiment, may also be provided separately or in any suitable sub-combination.

1. A system for deployment of a sensor in a blood vessel comprising:
a radially expandable sensor;
at least one anchor element attached to the sensor and configured to maintain the sensor in contact with the vessel wall during physiologic expansion and contraction thereof;
an expandable element configured within the sensor such that, in use, expansion of the expandable element causes the sensor to expand to radially fix the at least one anchor element in a wall of the blood vessel;
wherein the sensor is configured to produce a wireless signal correlated with the vessel diameter or area.

2. The system of claim 1 wherein the anchor comprises an erodible or fracturable element configured such that, upon fixing of the at least one anchor element in the wall of the blood vessel, erosion or fracture of the element decouples the sensor from the anchor.

3. The system of claim 2 wherein the erodible element is biodegradably erodible.

4. The system of claim 1 wherein the at least one anchor element is moveable from a first position wherein the anchor is non-engageable with the vessel wall to a second position wherein the anchor is engageable with the vessel wall.

5. The system of claim 1 further comprising:
a sheath configured at least partially about the sensor, the sheath configured for protecting the expandable element from the at least one anchor element.

6. The system of claim 5 wherein the sheath is removable from about the sensor such that removal of the sheath moves the anchor element from the first position to the second position.

7. The system of claim 1 wherein the expandable element is a balloon.

8. The system of claim 1 wherein the expandable element is a mechanical device.

9. The system of claim 1 wherein the expandable element comprises a tube.

10. The system of claim 9 wherein the tube comprises non-rigid material.

11. The system of claim 9 wherein the tube comprises a plurality of incisions through the surface of the tube along its length.

12. The system of claim 11 wherein the incisions are straight or wherein the incisions are curved or wherein the incisions are “S” shaped or wherein the incisions are saw-tooth shaped.

13. The system of claim 12 wherein a region of the tube about the incisions is expandable upon application of a force to the tube.

14. The system of claim 13 wherein application of a force to the tube comprises applying a longitudinal pulling force to an internal surface of the tube.

15. The system of claim 13 wherein application of a force to the tube comprises applying a longitudinal pushing force to an external surface of the tube.

16. The system of claim 14 further comprising a tether element fixed to an internal surface of the tube.

17. The system of claim 16 wherein the longitudinal pulling force is applied to the tether.

18. The system of claim 15 wherein the tube comprises a first inner tube and a second outer tube.

19. The system of claim 18 wherein the longitudinal pushing force is applied to the second outer tube.

20. The system of claim 1 wherein the at least one anchor element comprises a harpoon, a fish hook, a barb, a corkscrew or scales.

21. The system of claim 1 wherein the sensor is coil or crown shaped.

22. The system of claim 1 further comprising an anchor element attached to the sensor between a first and second end of the sensor.

23. The system of claim 22 wherein the at least one anchor element is attached to the first end of the sensor.

24. The system of claim 23 further comprising at least a second anchor element wherein the at least a second anchor element is attached to the second end of the sensor, opposite the first.

25. The system of claim 1 wherein the sensor is configured to obtain an area measurement of the blood vessel.

26. The system of claim 1 wherein the blood vessel is a vein.

27. The system of claim 23 wherein the vein is one of the jugular vein, the superior vena cava or the inferior vena cava, IVC.

28. A method for deployment of a sensor in a blood vessel comprising:
inserting a radially expandable sensor into a blood vessel, the sensor comprising at least one anchor element;
expanding an expandable element configured within the sensor such that expansion of the expandable element causes the sensor to expand to radially fix the at least one anchor element in a wall of the blood vessel;
removing the expandable element from within the sensor; and
receiving a wireless signal correlated with diameter or area of the blood vessel from the sensor;
wherein the anchor element maintains the sensor in contact with the vessel wall during physiologic expansion and contraction of the blood vessel.

29. The method of claim 28 further comprising:
removing a sheath from the sensor prior to expanding the expandable element.

30. The method of claim 28 wherein the blood vessel is a vein.

31. The method of claim 28 wherein the vein is one of the jugular vein, the superior vena cava or the inferior vena cava, IVC.
